(19) United States
(12) Patent Application Publication
(10) Pub. No.: US 2026/0294283 A1
(43) Pub. Date: Oct. 1, 2026

(54) NOVEL DYNAMIC HEARING RESTORATION DEVICE

(71) Applicant: MELISONO AB, Lund (SE)

(72) Inventor: Bernt BÖHMER, HÖÖR (SE)

(21) Appl. No.: 19/475,896

(22) PCT Filed: May 2, 2024
(86) PCT No.: PCT/SE2024/050420

§ 371 Date: Oct. 16, 2025

(30) Foreign Application Priority Data
May 8, 2023 (SE) 2350562-1

(51) Int. Cl. A61B 5/12; A61B 5/00

(52) U.S. Cl. CPC A61B 5/123; A61B 5/725

(57) ABSTRACT

The present invention discloses a dynamic hearing restoration device comprising a software unit for offline and/or online processing of compensating data obtained from a hearing assessment and/or correction test, said software unit for offline and/or online processing arranged for obtaining data from a test object, for processing and compensating collected obtained data from the test object by identifying and selecting candidate correction frequencies where correction gain could be applied, by performing a method involving the steps of:—finding peak frequencies where required correction gain is higher than neighboring frequencies in the obtained data, and—preferably then calculating a weight of every identified peak based on a distance to a nearby located peak and the correction gain of each identified peak, wherein the weight of every identified peak is a multiplication of the distance to the next peak and the correction gain of the peak.

### INTRODUCTION

Traditional hearing restoration devices, hearing aids etc., relies on the ISO standard hearing threshold test involving discovery of a person's hearing threshold at a handful of frequencies, normally about 6 to 10. The result of the test is recorded in an Audiogram. Whilst the test is useful for detecting a person's hearing loss through the uncovering of an elevated hearing threshold it is not revealing any information about hearing loss above the hearing threshold. Any hearing restoration device that is applied based on the standard test must rely on the assumption that hearing loss is linear at higher sound pressure levels and more or less equal to the elevated threshold. However, a novel Equal Loudness Hearing Test clearly reveals that this is not the case. The hearing ability of a person experiencing hearing loss changes significantly at sound pressure levels above the hearing threshold.

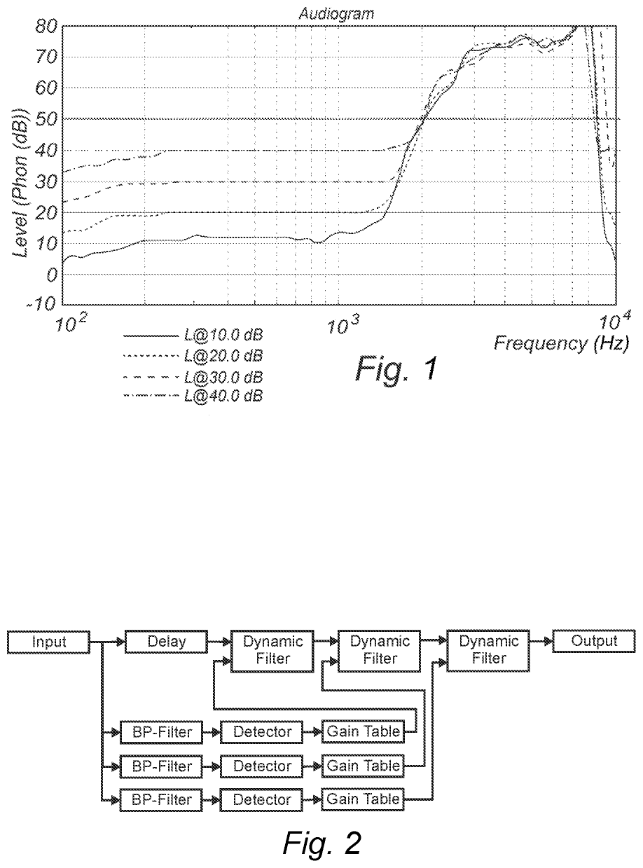

FIG. 1 shows Equal Loudness Hearing Test data at levels 10 Phon, 20 Phon, 30 Phon and 40 Phon for a person with quite significant high frequency hearing loss. The bottom trace shows the lowest 10 Phon level which is just above or at the test subject's hearing threshold at 800 Hz. The traces display to the right a significant elevation of the hearing threshold starting just above 1 kHz. To the left in the diagram, at lower frequencies, the traces are spaced by the expected 10 Phon. At higher frequencies beginning from approximately 2 kHz and above, the spacing is significantly less than 10 Phon. At 300 Hz the 10-40 Phon traces are separated by almost 40 dB, which corresponds to the sound level increase. The slight elevation of the 10 Phon trace between 100 Hz-1 kHz produces a slightly smaller separation to the 20 Phon trace than between the other traces. This is caused by a slight elevation of the hearing threshold influencing at the lowest 10 Phon level. The elevation disappears at 20 Phon and above where the spacing is exactly 10 Phon apart from a few small local deviations. At higher frequencies above 2 kHz, where there is significant elevation of the hearing threshold, the 10-40 Phon traces are separated by just a few Phon. This reveals that at say 3 kHz the sound pressure level just needs to increase by a couple of Phon to be perceived by the test subject to have increased by 40 Phon. Very clearly the brain starts to radically compensate for the elevated hearing threshold once the sound pressure level is above the hearing threshold and the ear/brain is no longer behaving as a linear device, clearly invalidating the conventionally accepted assumption that hearing loss is linear.

This behavior will create a massive problem if the elevated hearing threshold were to only be compensated by the present standard methods relying on the assumption of linear hearing behavior. Using standard compensation practice, applying static amplification of approximately half to two thirds of the measured elevated hearing threshold, the sound would at lower sound levels and frequencies above approximately 2 kHz evidently not be amplified enough and at higher levels amplified far too much. This is further evidenced by the fact that the test subject whose hearing is measured with Equal Loudness Hearing Test and shown in FIG. 1 has got professionally fitted hearing aids but is not using them since they do not provide any perceived benefit.

Normally the dynamic range compression at frequencies with an elevated hearing threshold is smaller than the case presented in FIG. 1, but all measured test subjects show similar dynamic compression at frequencies where there is an elevation of the hearing threshold. Looking at our collected measurement data it is obvious that the brain automatically compensates for an elevated hearing threshold at higher sound pressure levels when the sound is above the hearing threshold and consequently can be heard. The brain's compensation can be quite dramatic as in FIG. 1 or smaller, but it is always present to a significant degree.

To be able to properly restore hearing a device clearly cannot rely on static amplification, dynamically changing amplification depending on sound level is required, and Equal Loudness Hearing Test data is necessary to set correct amplification at different sound levels. Output from the Equal Loudness Hearing Test is amplification data tables at selected filter frequencies. The table data is used in the Dynamic Hearing Restoration Device as input to set the dynamically changing amplification at each frequency depending on sound level.

Apart from the most important aspect, enabling a proper hearing restoration, a Dynamic Hearing Restoration Device also utilizes available dynamic range in an efficient manner. The current practice hearing compensation based on static amplification, consumes a significant portion of the available dynamic range without any benefit. The static amplification approach is in fact exposing the user for a risk of acquiring additional hearing loss due to the disadvantageous and unnecessary exposure to sound pressure levels higher than required. If the standard test reveals an elevated hearing threshold of approximately 70 dB at 3 kHz, as is the case for the person with hearing loss according to FIG. 1, a normal approach would be to amplify sounds at that frequency by about halt to two thirds of the measured elevation. In this case it would be about 35 dB to 45 dB, let us say 40 dB is used.

Shown by Equal Loudness Hearing Test results in FIGS. 1, 40 dB amplification is not going to be enough at sound levels below 30 Phon, it is exactly the right amplification for sounds at 30 Phon but at higher sound levels it is too much. At approximately 70 Phon, apparent by the Equal Loudness Hearing Test data in FIG. 1, no amplification is required anymore which makes the static 40 dB amplification incredibly excessive. It is thus no mystery why the person whose hearing measurements are shown in FIG. 1 is not helped by the traditional approach.

With a Dynamic Hearing Restoration Device, having dynamic amplification that changes depending on sound level, the user would not be subjected to any additional amplification at sound levels above 70 Phon. The Dynamic Hearing Restoration Device eliminates the risk of sustaining more hearing loss due to high sound levels inadvertently and unnecessarily being generated by the hearing loss compensation.

A Dynamic Hearing Restoration Device also utilize available dynamic range much better; as an example, a personal sound application in a mobile device with Bluetooth connected earbuds or headphones has already a quite limited dynamic range, limited both by headphone hardware and the Bluetooth connection. The digital lower limit is never better than 16 bit resolution i.e., 96 dB dynamic range and in practice together with hardware limitations it will be less. If 40 dB of that dynamic range is already used by static amplification, we only have at best 56 dB left. If we assume that the maximum output of the Bluetooth headphone hardware is 100 dB we would not be able to reproduce any sounds below 44 dB, which is a hard digital resolution floor not a noise floor. Literally no sounds softer than 44 dB can be reproduced, which means that quite a lot of softer sounds would be totally inaudible and defeat the purpose of the system i.e., restoring hearing for better intelligibility. The same situation applies in hearing aids, regularly operating with more limited bit depths. The Dynamic Hearing Restoration Device will not decrease the available dynamic range since amplification is lowered down to 0 dB at higher sound levels.

### SUMMARY OF THE INVENTION

The Dynamic Hearing Restoration Device uses data from a novel Equal Loudness Hearing Test that assesses hearing loss across a range of sound levels. The hearing loss data is received in the form of hearing data gain tables containing information about required amplification at each test frequency and different sound levels. The gain table format is just an example of a preferred way of communicating test results, many other data formats can of course be used.

According to a most general aspect of the present invention there is provided, a dynamic hearing restoration device comprising a software unit for offline and/or online processing of compensating data obtained from a hearing assessment and/or correction test, said software unit for offline processing arranged for obtaining data from a test object, for processing and compensating collected obtained data from the test object by identifying and selecting candidate correction frequencies where correction gain could be applied, by performing a method involving the steps of:
finding peak frequencies where required correction gain is higher than neighboring frequencies in the obtained data,
preferably then calculating a weight of every identified peak based on a distance to a nearby located peak and the correction gain of each identified peak, wherein the weight of every identified peak is a multiplication of the distance to the next peak and the correction gain of the peak.

Although optional, according to one embodiment of the present invention, the method comprises calculating a weight of every identified peak based on a distance to a nearby located peak and the correction gain of each identified peak, wherein the weight of every identified peak is a multiplication of the distance to the next peak and the correction gain of the peak.

The Equal Loudness test results shown in FIG. 1 uses 81 frequencies in the range between 100 Hz and 10 kHz. The Dynamic Hearing Restoration Device does not require this many correction filter frequencies. The number of correction filter frequencies can be anything between 1 up to the presented 81 or even more. Preferably correction filter frequencies should be 100 Hz, 200 Hz, 400 Hz, 800 Hz, 1200 Hz, 1600 Hz, 2400 Hz, 3200 Hz, 4800 Hz, 6800 Hz, 9600 Hz and 12800 Hz. These frequencies are evenly spread across the frequency range that is relevant for hearing restoration. They are more closely spaced at higher frequencies where higher resolution is required for optimal correction.

The Equal Loudness Hearing Test measurements are referenced to sound levels measured in Phon. If the measurements are not pre-processed before being sent to the Dynamic Hearing Restoration Device a transposition in the device of equal loudness data measured in Phon to sound pressure levels (SPL) is required before the data can be put into the Gain Tables, see FIG. 2. Input signals to a Dynamic Hearing Restoration Device from microphones or other sources are always referenced to physical sound pressure. Therefore, SPL referenced data is required for efficient signal processing in Dynamic Hearing Restoration Devices. SPL is a physical unit normally measured in dBSPL, which is a logarithmic unit using a reference level of 20 micro pascals of sound pressure, whereas Phon is a logarithmic perceptual unit. Perceptual levels measured in Phon are based on human perception of the loudness of sound.

The Equal Loudness Hearing Test measurements usually only covers a limited dynamic range, 10 Phon to 60 Phon. A Dynamic Hearing Restoration Device requires data for the whole relevant 0 dBSPL to 110 dBSPL dynamic range for proper hearing correction. If the data from the Equal Loudness Hearing Test is not covering this whole range, it is necessary to add missing hearing correction data by calculations in the device so that all required sound level entries in the Gain Tables can be filled with relevant gain information. Correction gain data for the missing portions of the dynamic range is calculated by polynomial extrapolation of the available measured range at all frequencies.

Next action involves selecting suitable correction frequencies where correction gain should be applied. Frequencies where there is no correction gain required according to the measurement are discarded. Frequencies where the hearing loss is too severe for correction to be feasible are also discarded. This typically happens at the highest frequencies, 9600 Hz and 12800 Hz, where age related hearing loss can make it impossible to hear the frequencies thus making correction pointless and even potentially harmful if too much gain is applied. Finally, the remaining correction gain data is analyzed to find peaks in the hearing loss data where it is suitable to apply correction. One peak frequency can usually cover a frequency range, due to the correction peaking filter bandwidth, which often makes it unnecessary to boost adjacent frequencies closest to the center frequency.

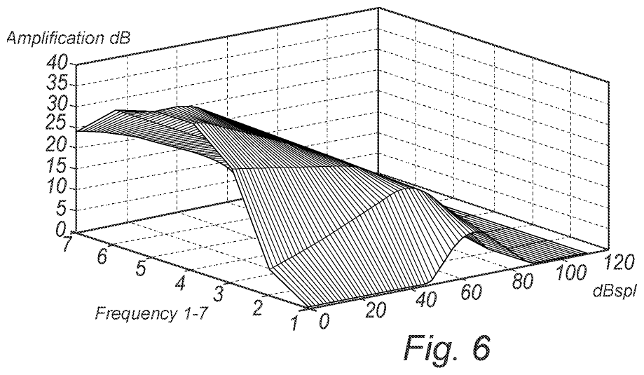

If the Equal Loudness Hearing Test measurements are not post processed to adjust Gain Table data for aggregate amplification, it must be done in the device before the data can be put into the Gain Tables. Amplification from adjacent Dynamic Filter frequencies interact and add together creating additional gain. Dynamic Filter bandwidths must be wide to achieve good time domain response with limited energy dispersion i.e., ringing. The wide bandwidth consequently causes an overlap between adjacent Dynamic Filter frequency bands. To correct aggregate gain to equal the measured required gain an optimization must be performed. The optimization is termed gain sail optimization since the applied gain looks like a sail in a three-dimensional plot with frequency, sound level and amplification on the axes, see FIG. 6.

The Dynamic Hearing Restoration Device contains correction peaking filter blocks at the minimum at each correction frequency providing dynamically varying amplification dependent on the sound level. FIG. 2 shows a high-level topology with three dynamic filters, each handling amplification at one frequency. The number of filters could be fewer or more depending on the number of frequencies where amplification is required, but the general principle should be clear. Input could be from a digital and/or analog stream of sounds and/or one or more microphones. The sound output signal from the Dynamic Hearing Restoration Device is normally sent to a sound transducer of some sort either in the device or at the end of a signal path and/or could be recorded for later playback. Typically, the Dynamic Hearing Restoration Device is in the form of hearing aids, earbuds or headphones.

The elements required for the whole dynamic amplification mechanism greatly influences sound quality. Human hearing is very sensitive to time domain anomalies and the dynamic amplification changes must be achieved without audible artifacts being introduced. Audible sound degradation would indeed defeat the purpose of the device, to restore hearing. Therefore, sound level detector filters, detector properties, amplification filter bandwidths and the method used to realise the amplification changes have to be carefully chosen. The invention takes all these aspects into account and the revealed dynamic amplification generates no sound degradation.

Signal processing in the Dynamic Hearing Restoration Device should preferably be software algorithms in a DSP or analog or a combination of the two. The corresponding sound pressure level must be known and calibrated inside the Dynamic Hearing Restoration Device when amplification is applied since the amount of amplification that should be applied is dependent on the sound pressure level.

A typical implementation also requires a limiter applied on the output to reduce maximum sound pressure. The limiter will prevent risk of additional hearing damage by keeping reproduced sound pressure within safe limits and if known, limit levels to stay within the output transducers's dynamic reproduction range.

If the input signal source comes from one or more microphones in a real time processing application of the Dynamic Hearing Restoration Device, typically in the form of hearing aids, earbuds or headphones, acoustic feedback cancellation is also required.

### DETAILED DESCRIPTION OF THE INVENTION

If the hearing correction data provided by the novel Equal Loudness Hearing Test to the Dynamic Hearing Restoration Device is not post processed it will be referenced to Phon. It will also likely have a limited dynamic range, normally 10 Phon to 60 Phon. Further, if not already done, suitable correction frequencies must be identified and selected before data can be entered into the Gain Tables of the device, see FIG. 2. These steps need to be carried out in the device if not already preprocessed before the data is received by the device or it could be sent to a processing software running on an external device such as a mobile phone, PC, cloud processing unit or other similar alternatives.

Sound pressure level measured in dBSPL is a logarithmic unit using a reference level of 20 micro pascals of sound pressure i.e., 0 dBSPL equals a physical sound pressure of 20 micro pascals. The ISO226 standard provides perceptual equal loudness information at different sound levels and frequencies. Perceptual levels are measured in Phon, which is a unit based on human perception of loudness. As an example, two tones, both at a level of 30 Phon, at the frequencies 1 kHz and 100 Hz respectively, will be perceived to be equally loud by a person with normal hearing ability. The tone at 100 Hz will however need to be at a higher physical sound pressure level compared to the tone at 1 KHz for them to be perceived as equally loud.

The equal loudness test's sound levels are based on the ISO226 standard and are measured in Phon. Since Phon is a perceptual level, each frequency test point and sound level must be translated from Phon to a technically derivable physical sound pressure measured in dBSPL before it can be used in the Dynamic Hearing Restoration Device's Gain Tables. Input signals to a Dynamic Hearing Restoration Device from microphones or other sources are always referenced to physical sound pressure and consequently SPL referenced gain data is required for accurate signal processing.

The ISO226 standard only contains a finite number of numerical test points divided in frequency in a one third octave series from 20 Hz to 12 500 Hz. The number of numerical level test points are similarly limited.

The equal loudness test's chosen frequency points do not fully coincide with the ISO226 frequencies and the level data available in the ISO226 standard has a granularity that is too coarse for it to be useful directly. Consequently, the required data in between the numerical test points available in the standard must be interpolated. There are many possible mathematical interpolation methods that can be used, in this case the interpolated values are determined by cubic spline interpolation. Both level and frequency plane data must be interpolated to arrive at the required granularity.

Then, using the ISO226 interpolated data, the transposition from Phon to dBSPL as reference for hearing correction gain can be done. Measured correction gain data is interpolated from Phon reference to dBSPL reference. Again, there are many possible mathematical interpolation methods that can be used, in this case interpolated dBSPL referenced values are determined by cubic spline interpolation. Correction gain is measured in logarithmic units, dB, and not in perceptual Phon.

A Dynamic Hearing Restoration Device must handle a dynamic range from 0 dBSPL to 110 dBSPL to accomplish proper hearing correction. Therefore it is necessary to extend the measured hearing correction data to cover the whole applicable dynamic range. Below 0 dBSPL the same correction gain as at 0 dBSPL will be used and at nearly all frequencies the level is below the hearing threshold anyway. Similarly, levels above 110 dBSPL have the same correction gain as at 110 dBSPL and such high levels will not be amplified in any case, they are attenuated by a maximum level limiter.

The Equal Loudness Hearing Test normally provides data from 10 Phon to 60 Phon in 10 Phon increments at each frequency. After the Phon to dBSPL transposition, data is available referenced to physical dBSPL units. Sometimes the available level range is smaller but a minimum of two and normally four to six level measurement points are available.

At first, the level granularity is increased. Interpolation can be achieved mathematically in many ways, in this case cubic spline data interpolation is employed between the raw data points followed by a multiple order linear phase FIR average filtration of the interpolated data set. Filtration is made to smooth out local variations in the measured data to achieve an improved accuracy. The interpolation can generate as many datapoints over the available dynamic range as desired, in this case a 0.5 dB interval between data points is used.

Secondly, the dynamic range is extended below the lowest measured level down to 0 dBSPL. For this purpose, the lowest levels of the interpolated and filtered data are used. A straight-line, first order polynomial, derivative fit is made to the derivative of the lower level part of the interpolated and filtered data. The first order polynomial is then used to extend the data below the lowest measured level. Similarly, the interpolated and filtered data at the highest measured levels is used to perform a first order polynomial fit to the derivative of the highest level part of the interpolated and filtered data. The resulting first order polynomial can then be used to extend data points above the highest measured level. Finally, a multiple order linear phase FIR average filtration of the entire extended data set is made. Filtration smooths out the transitions between measured and extended data to improve accuracy. FIG. 6 displays a 3D diagram with measured and extended correction gain data, called a gain sail, at seven different frequencies.

Next step involves identifying candidate correction frequencies where correction gain could be applied. Frequencies where the equal loudness test shows that correction gain is not required will not be used and are discarded. Frequencies where the equal loudness measurement shows that hearing loss is too severe for correction to be applied are also discarded. The indicators being that excessive correction gain is required to restore hearing or simply that the test was terminated because the sound level was too loud. It is not unusual that the highest frequencies, 9600 Hz and 12800 Hz, pose problems for people with significant age related hearing loss, making it impossible for them to hear these frequencies. The remaining candidate frequencies are then further analyzed to find the appropriate correction frequencies. First, correction peaks are located within the candidate correction frequencies.

Peaks in this case means frequencies where the required correction gain is higher than the neighboring frequencies. When the peaks are identified, each peak's “weight” is calculated. A peak's weight is a multiplication of distance to next peak and the peak's correction gain. Then, the dominant peak with the largest weight is added to the selected frequencies. This procedure is iterated to find more peaks among the identified peaks and new peaks are consecutively added to the selected frequencies if the frequency distance to an existing peak among the selected frequencies is larger than a threshold. A suitable threshold is a factor of two away from an existing frequency. When all identified peaks have been investigated the procedure stops, the correction gain frequencies are now selected.

The Dynamic Hearing Restoration Device contains one or more filter blocks providing dynamically varying amplification, each handling an individual frequency range. FIG. 2 shows an example filter topology that contains from the left BandPass-Filters that limits the frequency range before the level Detectors, level Detectors that detects the momentary input sound pressure level, Gain Tables that uses table data to produce gain settings in the Dynamic Filters. A Dynamic Filter amplifies sound within the Dynamic Filter's frequency band according to Gain Table data input.

The filter block can of course be built using topologies other than what is shown in FIG. 2 and the example is just one of many possible implementations.

Other means than the mentioned Gain Tables can obviously be used to determine the Dynamic Filter's sound pressure level dependent gain. It is easy to understand that a polynomial or some other type of mathematical expression can be used as a substitute and the Gain Tables merely serve as an example of a feasible implementation approach. The Gain Tables shown in FIG. 2 would in such case be substituted by the applied alternative method used to obtain the Dynamic Filter gain.

A Dynamic Filter is required at each frequency that needs to be amplified within the hearing restoration process. The Dynamic Filter is operating with dynamically changing gain dependent on the dynamically changing input signal level at the filter frequency. Many basic filter topologies are conceivable for the Dynamic Filter. For example, IIR topologies, FIR topologies etc. or combinations thereof. Regardless of the type of filter there is always a direct relationship between filter bandwidth and time domain response. A filter with narrower bandwidth will have poorer time domain response compared to a filter with wider bandwidth. As the bandwidth of the filter is reduced, progressively there will be more ringing present on the filter output. Also, the output response after a transient input signal to the filter will be more sluggish and delayed by a narrower bandwidth filter than a wider band filter. The filter order will also have a similar influence on the time domain response, where higher order filters produce better stopband attenuation or passband gain but exhibit poorer time domain behaviour.

The Equal Loudness Hearing Test provides data about required amplification at a selected number of frequencies and sound levels to restore a test subjects hearing. The data is provided in the form of tables, one table for each frequency. The tables collectively, see FIG. 6, creates a gain sail. FIG. 6 displays a three-dimensional diagram with gain sail data from a person facing cookie bite hearing loss. The gain sail shows the required correction gain (amplification) that is required at the frequencies 1-7 at sound pressure levels from 0 dBSPL to 120 dBSPL. The frequencies in this case are 400 Hz, 800 Hz, 1200 Hz, 1600 Hz, 2400 Hz, 3200 Hz and 12800 Hz. The gain sail reveals the measured correction gain required to fully restore hearing for the test subject and is a collection of correction gain table data for each frequency. Looking at the gain sail, it is apparent that at higher sound pressure levels no correction gain is required, but at lower levels significant amplification is. Above approximately 80 dBSPL no amplification is necessary at any of the frequencies, whereas at 20 dBSPL upwards of 35 dB to 40 dB is needed in the middle frequencies to correct the measured hearing loss.

To restore hearing loss a Dynamic Hearing Restoration Device must dynamically change filter gain at the 7 frequencies depending on the input sound pressure level at each of the frequencies. A topology is shown in FIG. 2 containing three filters that handle dynamic amplification at three different frequencies. The Dynamic Hearing Restoration Device must, in this example, comprise 7 band pass filters that predominantly pass each of the frequencies separately to 7 sound pressure level detectors, one for each frequency. The level detector outputs are then used to calculate the momentarily required gain of 7 peaking filters, again one filter for each frequency. The number of necessary frequencies varies from case to case and the 7 in this case is only an example. In some cases just one frequency is required but in most cases two to five is enough. In theory, all twelve of the normally available frequencies could be needed but it is unusual.

There are two obvious high-level topologies for a Dynamic Hearing Restoration Device. A serial topology or a parallel. FIG. 4 shows an example of a serial topology and FIG. 5 reveals a parallel topology. Many combined Filter Block compositions can of course be constructed using other topologies than those shown in FIGS. 4 and 5. A serial topology is however preferred as it has got some signal processing advantages. All minimum phase filters produce both a magnitude deviation and an associated phase deviation. With a serial topology it is straight forward to add gain at consecutive frequencies. Any added filter gain magnitude regardless of phase angle will always be present on the output. With a parallel topology each individual filter's gain magnitude will not necessarily be produced at the output. The output signals from filters in parallel are not necessarily in phase at all frequencies, their respective output's phase changing as more or less boost is applied. This will cause somewhat unpredictable results since the summation of the parallel filters is not only a magnitude summation but a vector summation of each output producing different magnitudes depending on their phase relationships. Consequently, for simplicity and effective use of energy and computational power in a real time application, the serial topology is much preferred. Typically, energy is in very short supply in wearable earbuds, hearing aids or similar products.

FIG. 2 shows a serial filter topology which is combined with a parallel detector topology. The parallel detector paths make it possible to detect levels at each frequency without delay thereby avoiding time domain anomalies that could otherwise occur. The BandPass-Filters filter out relevant frequencies before the signal propagates to the Detectors. The Detectors detect instantaneous levels in each frequency band. The Detectors have a short rise time, between 1 us to 100 us, and a somewhat longer fall time between 100 us to 2 ms. The values are audibly optimized to remove perceptible time domain anomalies. The outputs from the detectors are then sent to the Gain Tables. Based on the current sound levels desired gain values are found in the tables and then output from the Gain Tables to the Dynamic Filters. The input and output signal must be level calibrated, so that the corresponding sound pressure level is known. The Delay block in the signal path is used to delay the audio signal for a short period before it is passed to the Dynamic Filter chain. The delay should correspond to the response time of the Detectors and Dynamic Filters. If the signal level is very low, the Dynamic Filter gain is usually high and, in some cases, where a lot of amplification is required for hearing correction, it can become very high. If the signal level rises very quickly, as an example if a glass object falls on a hard floor, the gain must be reduced very quickly, or it will momentarily be too high. The Delay element is used to balance the dynamic behavior so that the high sound level propagates through the Dynamic Filter at the same speed as the gain is adjusted. Without the Delay element, the gain will be momentarily too high when the signal level rises very quickly, which will produce an annoying clicking type of sound. This behavior can be avoided using the Delay element.

Building Dynamic filters in the digital domain using standard building blocks whilst preserving signal fidelity is not straight forward. The ubiquitously used infinite impulse response, IIR, and finite impulse response, FIR, filters do not behave predictably and well with dynamically changing filter coefficients. Nor does any combination of these filters. An IIR filter, regardless of chosen DSP implementation topology, operates through nested feedback paths with different delay lengths and coefficients used throughout for multiplications. For example, the common IIR bi-quadratic building block comprises five coefficients and four delay elements holding data from previous sample cycles. Any input signal other than zero to an IIR filter produces an impulse response on the output that tails off over time. The tail time is by definition infinite but in practice the output response will eventually fall into the noise floor, either through round of errors in the DSP-calculation's bit depth limited precision or an actual noise floor present in the input signal. The length of the tailing signal depends on the chosen filter order and Q, higher order and higher Q filters having longer tails. A FIR filter has within its filter structure similar delay elements holding data from previous samples and a multitude of coefficients. The filter does not rely on nested feedback loops, it uses a finite number of delay elements and coefficients defined by the filter length. The finite length, number of samples, of the filter also limits the tailing response to the same finite length of samples. Although a finite tailing response could sound beneficial, a significant drawback with FIR filters compared to IIR is the latency they introduce. A FIR filter always delays the output signal by its number of samples finite length, which could be very troublesome in real time applications such as a Dynamic Hearing Restoration Device if the filter is long. Humans are sensitive to latency, even a few tenths of milliseconds is clearly perceptible and sounds that doesn't occur at the correct time in relation to visual impressions produces a quite strange and confusing feeling which is unacceptable for the application.

The tail on the output of any filter after the input signal has become zero is a measure of the energy stored in the delay elements within the filter. The filter models and formulas used to calculate filter coefficients from parameters assumes that the filter structure initially is in a steady state of zero, which, after an input signal has gone to zero, it only reaches after processing possibly hundreds of samples due to the nested feedback paths with different delay lengths or the filter length in case of a FIR filter. Regardless of whether it is an IIR, or FIR filter the stored energy within the filter is similar and the number of samples required to reach steady state of zero within the filter is more or less the same.

In a Dynamic Hearing Restoration Device, filters will never reach steady state zero within themselves since there will always be an input signal present. Filter parameter updates will always be done whilst energy is present within the filter structure. If filter parameters were updated occasionally, say once every ten seconds, this would not be a major issue, but a dynamic filter must be updated much more often, potentially every sample. Updating coefficients at a rate not far removed from or even approaching the sample rate will produce significant distortion. Updated coefficients will be multiplied with old data stored in the filter and consequently the output from the filter will become erroneous for as long as it takes for the stored data to propagate out through the filter structure. If coefficients are updated every sample the distortion will become very substantial.

Filters built on the Digital Integrator Cascades technique developed by Hal Chamberlin are an alternative to the IIR and FIR filters in dynamic applications. These filters are better suited to dynamic updates with much less distortion, but the filter responses are less accurate when resonances are introduced. The basic integrator form of these filters can only produce a limited set of filters and second order filters with Q values i.e., resonances, are not as accurate. Therefore, although commonly used in real time dynamic applications such as gaming software, their properties are less ideal in a hearing restoration system.

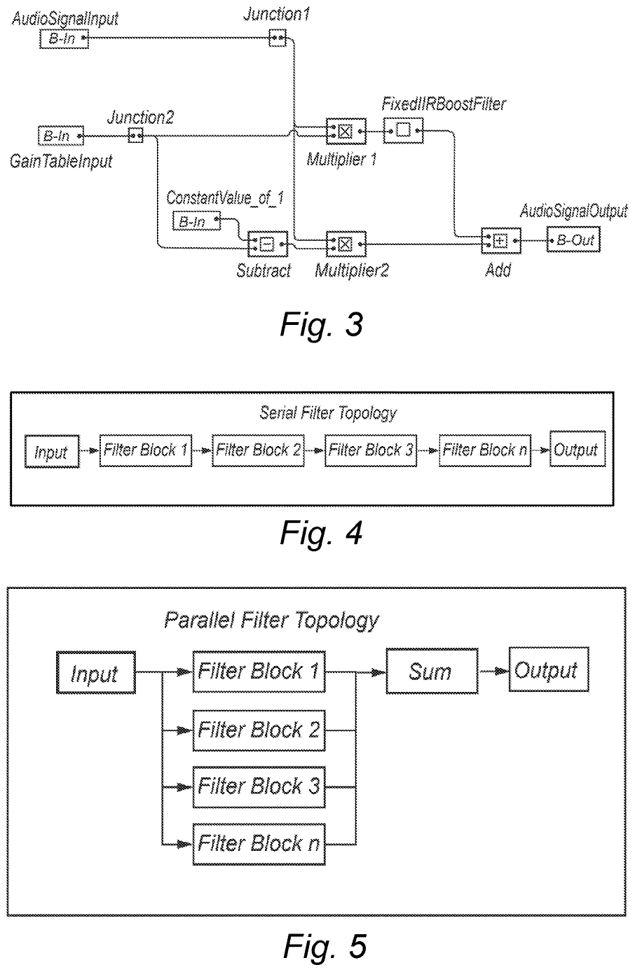

FIG. 3 shows a feasible implementation of the Dynamic Filter block present in FIG. 2 using an alternative structure that does not possess the drawbacks caused by dynamic updates of filter coefficients discussed above. The structure uses a fixed IIR filter, FixedIIRBoostFilter, that adds gain at the desired boost frequency. This filter can obviously be substituted with another type of filter or a combination of filters, the IIR filter is only a suitable example. The filter's coefficients are statically set so that maximum required boost is always provided by the filter. As an example, the gain sail in FIG. 6 shows that a maximum gain of approximately 35 dB is required at frequency 5 and consequently the filter gain would be set to 35 dB in this case. To the left in FIG. 3 are the input signals to the Dynamic Filter structure, AudioSignalInput and GainTableInput. The AudioSignalInput is obviously the audio input signal and the GainTableInput is the gain control input to the Dynamic Filter. The gain control input signal varies between zero and one depending on the required dynamically changing gain. When the signal is one, the Dynamic Filter produces maximum gain on the output and when zero it just passes through the audio signal without any gain at all being applied. The gain control signal is fed to Multiplier1 that is multiplying the audio input signal with the gain control signal. The gain control signal is also after being subtracted from the constant value of one fed to Multiplier2. The gain control signal after the subtraction varies between one and zero i.e., when the gain control signal to Multiplier1 is one the signal to Multiplier2 is zero and when zero to Multiplier1 it is one into Multiplier2. The audio signal output from Multiplier1 is fed to the FixedIIRBoostFilter which applies maximum gain and the output from the FixedIIRBoostFilter is fed to an adder, Add, summing the signal with the output form Multiplier2. The sum of the audio signal outputs from Multiplier1 and Multiplier2 will always add up to the same as the input audio signal, the ratio however between what goes out of Multiplier1 and Multiplier2 changes depending on the gain control input signal to the Dynamic Filter. By changing the gain control input signal to the Dynamic Filter a different mix of the unmodified but gain controlled output from Multiplier2 is added together with the boosted output from Multiplier1. By changing the signal ratio through the gain control input signal to the Dynamic Filter the boost provided by the Dynamic Filter can be varied from zero to maximum boost. This is achieved through changes of a simple mix of two audio signals entirely without any added distortion.

FIG. 8 shows frequency responses of 8 Dynamic Filters with suitable bandwidths. Gain overlap between these filters can be seen in the diagram. Significant overlap occurs, especially in the middle frequencies that are relatively closely spaced. Close spacing is necessary to attain a correction that fits well with any measured hearing loss. The relatively wide bandwidth of the filters is also necessary, wide bandwidth filters have good time domain behavior whereas narrow bandwidth filters produce poor time domain response.

Good time domain behavior is required, human hearing is very sensitive to time domain behavior of sounds. If sound is appearing from drumming on wood or metal, a violin or trumpet being played are all interpreted by human hearing from the differences in time domain properties of the sound. A Dynamic Hearing Restoration Device obviously cannot be allowed to introduce time domain irregularities that would diminish sound quality making it harder to hear and interpret sound. Consequently, Dynamic Filters must have wide bandwidths to maintain sound quality and they will for that reason always have overlapping responses as exemplified in FIG. 8.

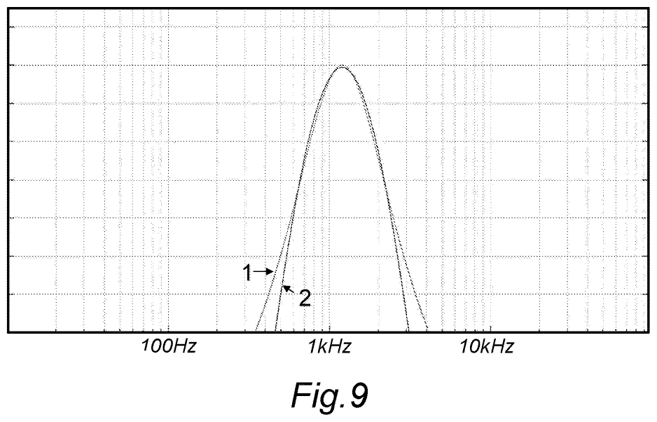

To achieve good tracking between dynamically applied correction gain and momentary sound pressure the Dynamic Filter bandwidth and the level Detector BandPass-Filter must have comparable frequency responses. FIG. 9 shows a Dynamic Filter frequency response at 1200 Hz, trace 1, overlapping a suitable Detector BandPass-Filter at 1200 Hz, trace 2. The bandpass filter cannot be too narrow as it would produce a poor time domain response and therefore not track the monetary sound pressure well. If the time domain tracking is bad, the measured momentary sound pressure would be incorrect and so would the applied correction gain that is obtained from the measured sound pressure. The Detector BandPass-Filter bandwidth cannot be much wider than the Dynamic Filter either as this would also produce incorrect sound pressure level measurements. With a wide band filter, sounds at frequencies far from the center frequency would be given too much weight in the measurement and gain would consequently be reduced at the center frequency. This is obviously wrong since the sounds are not present at the center frequency and the applied gain at the center frequency would become too low. The best results from a sound quality point of view are obtained when these two filters have similar frequency responses, as exemplified in FIG. 9.

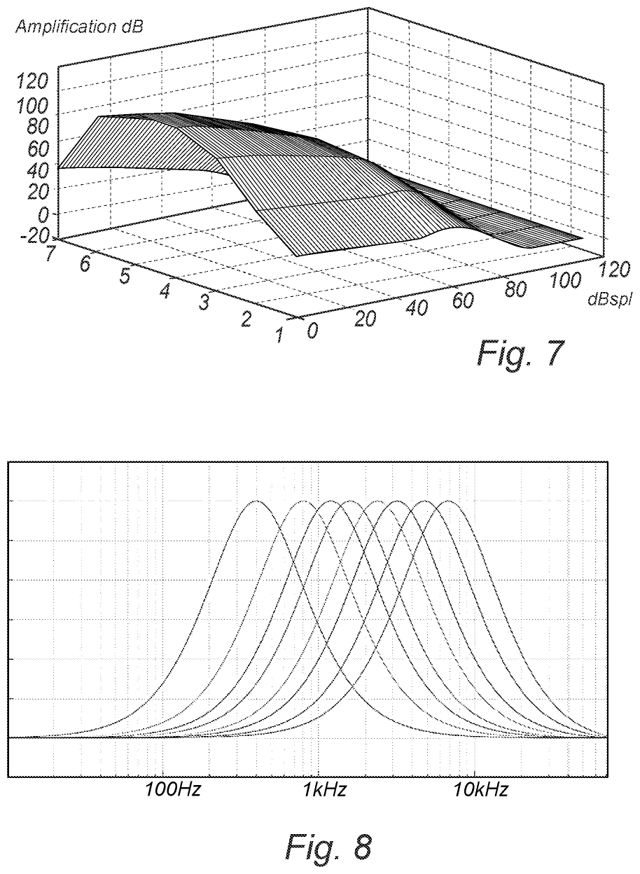

Employing filters with bandwidths shown in FIG. 8 to correct the measured hearing loss shown by the gain sail in FIG. 6 without taking gain overlap between the filters into account would produce far too much amplification. The gain sail in FIG. 7 displays the resulting total amplification if gain overlap was not considered. It is abundantly clear that the aggregate Dynamic Filter amplification without factoring in gain overlap produces far too much gain, upward of +90 dB in the middle frequencies where it should only have been around +35 dB.

Whilst it would be possible to correct for over gain with a feedback correction network in a real time signal processing Dynamic Hearing Restoration Device, a feedback network will always produce time domain anomalies which are detrimental to sound quality. The severity of the problem with feedback becomes quite clear when one considers that with a low input sound level, say 20 dBSPL, the aggregate amplification rises to +90 dB. Then, as an example, when someone starts speaking and a significant input sound level arises from silence, the gain must instantly be turned down to a much lower level, looking at FIG. 6 probably around 25 dB. The gain then needs to change 65 dB within much less than 10 ms, which will cause significant distortion of the sound. However, not only will significant distortion be generated but the worst problem is gain miss tracking. The gain will initially be too high and sounds that are rather low in level will inadvertently be reproduced at a very high level causing severe initial transient overshoot. Soft sounds will sound as if they initially were almost like gunshots before the gain is turned down, which is obviously not satisfactory. With such large gain adjustments over short periods of time gain tracking miss tracking will always be present. Feedback would also require additional real time processing steps that unavoidably consume processing bandwidth and energy.

There is, however, a better alternative to manage over gain which is termed gain sail optimization. With a Detector BandPass-Filter bandwidth that matches the Dynamic Filter bandwidth it is possible to preprocess gain table data and optimize gain sail amplification, thereby avoiding significant distortion, gain miss tracking causing gunshot like issues and unnecessary real time calculations. Matching Dynamic Filter and Detector band pass filter bandwidths are required for this to be possible so that the detector senses the sound level equal to how gain is applied by the Dynamic Filter. If the filter bandwidths are different preprocessing output will not be accurate.

Gain sail optimization is a mathematical problem that can be solved either using equations for an analytical solution or numerical iterative methods. Whilst an analytic solution is theoretically possible, given the complexity and very large number of variables and equations required would make such a solution wholly unfeasible. A numerical solution is therefore much preferred and a numerical iterative method to optimize the correction gain data before storing it in the Dynamic Hearing Restoration Device's Gain Tables is used.

Table data from the Equal Loudness Hearing Test, exemplified in FIG. 6, is used as target for the aggregate correction gain at each frequency and sound pressure level involved. The gain sail optimization aims to remove excess correction gain accumulated by adjacent frequency Dynamic Filter boosts. The optimization is done through least squares numerical optimization of filter gain.

When the least squares optimization has found the best correction gains at all levels and frequencies, the adjusted values are saved in the Dynamic Hearing Restoration Device's Gain Tables. The gain sail optimization can be done both before downloading the data into the Dynamic Hearing Restoration Device or through algorithms running inside the Device.

All modern hearing aids use feedback reduction techniques of various kinds. An aggressive feedback reduction system risk producing audible irregularities reducing the sound quality of the hearing aid. The more aggressive the greater the risk. The Dynamic Hearing Restoration Device's natural reduction of gain at higher levels put less burden on the feedback reduction system and it does not need to be as aggressive as would be required in a system with constant gain.

Exemplified by FIG. 6, required correction amplification will decrease when sound levels increase. This dynamic reduction of amplification at higher input levels provides a clear benefit in hearing aid and similar applications where there always is a risk of acoustic feedback. If there is feedback and the level at the feedback frequency increases, the amplification will automatically be reduced consequently reducing feedback, and limit feedback sounds to a low level. In comparison, a fixed gain system will just increase the feedback sounds until they reach maximum output from the system which obviously can be very unpleasant and need to be aggressively prevented and avoided. If acoustic feedback occurs in a Dynamic Hearing Restoration Device, amplification can simply be reduced a little bit at the level where feedback occurs until it stops. No aggressive methods are required since the feedback sounds will inherently be at a low level.

In a Dynamic Hearing Restoration Device most likely basic acoustic feedback sensitivity is know beforehand and it is possible in advance to limit correction gain in the Gain Tables thereby removing or at least minimizing issues with acoustic feedback caused by excessive amplification. A maximum correction gain individual for each correction frequency that correlates with the Dynamic Hearing Restoration Device's acoustic feedback properties can beneficially be applied to the correction gain data. The limiting should preferably be applied before gain sail optimization, to make sure the applied limit values are never exceeded by aggregate adjacent frequency gain.

A final limiting step aims to reduce amplification at the highest sound pressure levels. Normally, amplification is not applied at high sound pressure levels since it is not required for hearing correction. As an example, the correction gain required to correct hearing loss for a test subject displayed in FIG. 6 does not incorporate amplification above approximately 80 dBSPL at any frequency. However, at 20 dBSPL upwards of 40 dB is required at several frequencies. The required correction gain displayed in FIG. 6 is very common, no gain is usually necessary at higher sound pressure levels even for individuals suffering from quite substantial hearing loss. In rare cases, when correction gain is present at high sound pressure levels, it is desirable to reduce the gain to avoid additional hearing damage. In this step correction gain is therefore gradually reduced to zero at levels above 90 dBSPL so that input level plus gain never produces an output exceeding 100 dBSPL. The two threshold levels 90 dBSPL and 100 dBSPL are selected to mitigate risk of additional hearing damage and can be adjusted to any other desired level.

A Dynamic Hearing Restoration Device requires a limiter applied on the output to reduce maximum possible output sound pressure. The maximum sound pressure level can be adjustable and changed to any level, however, it should normally never exceed 100 dBSPL. The limiter will prevent risk of additional hearing damage by keeping reproduced sound pressure within safe limits and limit levels to stay within the output transducers's dynamic reproduction range. The response time of the limiter should be very fast to always keep the sound pressure level below the limit. Different limit levels at different frequencies can also be used by the limiter, allowing more low frequency energy to pass whilst capping mid and higher frequencies.

The described Dynamic Hearing Restoration Device is not just suitable for applications like hearing aids, earbuds or headphones. It can be used together with any sound reproduction system. Such a system can use any type of loudspeaker or headphones like a phone, tablet, TV-set, headphone amplifier, HiFi system, or computer. It is also well suited for implementations within sound reproduction systems in cars where the relationship between system output and sound pressure at the listener's ears is predictable due to the generally fixed physical location of a person within the car cabin. The Dynamic Hearing Restoration Device will function properly if the output level at the listener's ear is known i.e., a certain sound reproduction system output produces a known sound pressure level experienced by the listener. All these arrangements are only provided as examples and many other possible implementation scenarios can obviously be envisaged. Most applications for the Dynamic Hearing Restoration Device will be real time applications. It is however perfectly possible to pre-process audio material with the Dynamic Hearing Restoration Device's compensation thereby creating a library of personally compensated audio.

Software functions, digital signal processing and algorithms can be implemented in many ways, from pure hardware implementations to pure software/firmware or a mix between the two. The DSP functions in the described invention use code written for a digital signal processor. The described algorithms that process input hearing data on the Dynamic Hearing Restoration Device is implemented in software running on the target device, but the software could of course be implemented to run on any computing system such as a personal computer, phone, tablet, or other device. It can also be implemented on a purpose-built target system perhaps resembling an audiometer or cloud computing resources.

### SPECIFIC EMBODIMENTS OF THE PRESENT INVENTION

Below there are provided some further embodiments of the present invention. According to a most general aspect of the present invention there is provided, a dynamic hearing restoration device comprising a software unit for offline and/or online processing of compensating data obtained from a hearing assessment and/or correction test, said software unit for offline and/or online processing arranged for obtaining data from a test object, for processing and compensating collected obtained data from the test object by identifying and selecting candidate correction frequencies where correction gain could be applied, by performing a method involving the steps of:
finding peak frequencies where required correction gain is higher than neighboring frequencies in the obtained data,
preferably then calculating a weight of every identified peak based on a distance to a nearby located peak and the correction gain of each identified peak, wherein the weight of every identified peak is a multiplication of the distance to the next peak and the correction gain of the peak.

As notable from above, the dynamic hearing restoration device comprises a software unit for offline and/or online processing. This implies that the software unit may be directed to either only offline processing or only online processing, or to a combination of the two. To give examples, online processing is typically implemented within an embedded DSP, and offline processing is typically used in cloud pre-processing or within a purpose-built embedded system. In addition to this, this software unit may also be connected to a software unit for real-time processing, which is further explained below.

According to one embodiment, the method also comprises identifying the dominant peak with the largest weight and adding the dominant peak to the selected frequencies. It should be noted that this step is preferred, but optional according to the present invention.

According to one embodiment, the software unit for offline and/or online processing is arranged for performing a transposition of obtained data from Phon to absolute dBSpl values by interpolating measured correction gain data from Phon reference to dBSpl reference, preferably as a first processing step.

Furthermore, according to yet another embodiment, the software unit for offline and/or online processing is arranged for extending the dynamic range below the lowest measured level and/or above the highest measured level, preferably by using a first order polynomial, more preferably proceeded by a multiple order linear phase FIR average filtration.

According to yet another embodiment, the software unit for offline and/or online processing is arranged for performing a gain sail optimization involving a compensation for adjacent filter gain contributions where there is an aggregation of amplification from adjacent filters. Moreover, according to one further embodiment, the gain sail optimization involves removing excess correction gain accumulated by adjacent frequency correction peaking filter boosts, preferably by performing least squares numerical optimization of filter gain.

According to one embodiment, the software unit for offline and/or online processing is arranged in a computer or a mobile device, such as phone or tablet, or in the cloud, or part of an embedded system, or arranged in a hearing device, such as a hearing assessment device or hearing correction device, e.g. provided in headphones, a headset or hearing aids. All headphones are possible here, such as over-ears or in-ears etc.

In line with the above, according to one embodiment there is disclosed a system comprising the software unit for offline and/or online processing according to above and a connected software unit for real-time processing.

According to one embodiment, the software unit for real-time processing is arranged in a hearing device, such as a hearing assessment device or hearing correction device, e.g. provided in earbuds, headphones, a headset or hearing aids, or a computer or a mobile device, such as phone or tablet, or in the cloud, or part of an embedded system.

Moreover, according to yet another embodiment, the software unit for offline and/or online processing and/or the software unit for real-time processing involve one or more dynamic filters for compensating the data, preferably multiple dynamic filters are involved for compensating the data, preferably a number of from 2-20 dynamic filters are involved for compensating the data, preferably each dynamic filter is operating with dynamically changing gain dependent on dynamically changing input signal level at the filter frequency. According to one embodiment, said one or more dynamic filters are dynamically changed based on an input-signal and at least one parameter, preferably said one or more dynamic filters change the amplification depending on a change in sound pressure. Moreover, according to one embodiment, input sound pressure is amplified so that the experienced sound level is fully compensated.

Furthermore, according to yet another embodiment, the software unit for offline and/or online processing and/or the software unit for real-time processing involve a digital signal processing (DSP) unit arranged for digital signal processing of amplification data and filter center frequencies, preferably by involving one or more filter blocks. In relation to the above it should be noted that the DSP unit suitably preferably is part of the real-time processing, such as is provided in a unit, e.g. as a microprocessor, in the headphones, but it may also be arranged in the software unit for offline and/or online processing according to the present invention.

According to one embodiment, each dynamic filter involved has a center frequency in a range of 100 Hz-12.8 KHz.

Furthermore, according to yet another embodiment, said one or more dynamic filters are wideband filters with low order, preferably second order, and wherein the method involves compensation for adjacent frequency filter boost to ensure a control of obtained aggregate gain.

Moreover, according to one embodiment, there is provided a dynamic filter for each frequency that needs to be amplified within the hearing restoration process.

Furthermore, according to one embodiment, said one or more dynamic filters is operating with dynamically changing gain dependent on a dynamically changing input signal level at a certain filter frequency.

According to yet another embodiment, the software unit for offline and/or online processing and/or the software unit for real-time processing involve a digital signal processing (DSP) unit using one or more filter blocks comprising a band pass filter, a sound pressure detector, and a dynamic filter. Again, as notable, the DSP unit may be part of the software unit for offline and/or online processing or the software unit for real-time processing, or may in fact be part of both units, i.e. both units may have DSP units.

Moreover, according to one embodiment, the band pass filter is arranged to filter out and the detector is arranged to measure the signal level at the dynamic filter frequency and suppress sound signals present at other frequencies. Furthermore, suitably the band pass filter has a low order, preferably second order, more preferably with Q below 1. Furthermore, according to one embodiment, the band pass filter and the dynamic filter has matching bandwidths.

According to yet another embodiment, the software unit for offline and/or online processing and/or the software unit for real-time processing involve a digital signal processing (DSP) unit using one or more filter blocks comprising a gain table which uses table data, e.g. from algorithms, and translates the current input level to a gain setting in the dynamic filter.

Furthermore, according to one embodiment, the software unit for offline and/or online processing and/or the software unit for real-time processing involves a digital signal processing (DSP) unit using multiple filter blocks.

1. A dynamic hearing restoration device comprising a software unit for offline and/or online processing of compensating data obtained from a hearing assessment and/or correction test, said software unit for offline and/or online processing arranged for obtaining data from a test object, for processing and compensating collected obtained data from the test object by identifying and selecting candidate correction frequencies where correction gain could be applied, by performing a method involving the steps of:
finding peak frequencies where required correction gain is higher than neighboring frequencies in the obtained data, and
preferably then calculating a weight of every identified peak based on a distance to a nearby located peak and the correction gain of each identified peak, wherein the weight of every identified peak is a multiplication of the distance to the next peak and the correction gain of the peak.

2. The dynamic hearing restoration device according to claim 1, wherein the method comprises calculating a weight of every identified peak based on a distance to a nearby located peak and the correction gain of each identified peak, wherein the weight of every identified peak is a multiplication of the distance to the next peak and the correction gain of the peak.

3. The dynamic hearing restoration device according to claim 1, wherein the method also comprises identifying the dominant peak with the largest weight and adding the dominant peak to the selected frequencies.

4. The dynamic hearing restoration device according to claim 1, wherein the software unit for offline and/or online processing is arranged for performing a transposition of obtained data from Phon to absolute dBSpl values by interpolating measured correction gain data from Phon reference to dBSpl reference, preferably as a first processing step.

5. The dynamic hearing restoration device according to claim 1, wherein the software unit for offline and/or online processing is arranged for extending the dynamic range below the lowest measured level and/or above the highest measured level, preferably by using a first order polynomial, more preferably proceeded by a multiple order linear phase FIR average filtration.

6. The dynamic hearing restoration device according to claim 1, wherein the software unit for offline and/or online processing is arranged for performing a gain sail optimization involving a compensation for adjacent filter gain contributions where there is an aggregation of amplification from adjacent filters.

7. The dynamic hearing restoration device according to claim 6, wherein the gain sail optimization involves removing excess correction gain accumulated by adjacent frequency correction peaking filter boosts, preferably by performing least squares numerical optimization of filter gain.

8. The dynamic hearing restoration device according to claim 1, wherein the software unit for offline and/or online processing is arranged in a computer or a mobile device, such as phone or tablet, or in the cloud, or part of an embedded system, or arranged in a hearing device, such as a hearing assessment device or hearing correction device, e.g. provided in headphones, a headset or hearing aids.

9. A system comprising dynamic hearing restoration device according to claim 1, and a connected software unit for real-time processing.

10. The system according to claim 9, wherein the software unit for real-time processing is arranged in a hearing device, such as a hearing assessment device or hearing correction device, e.g. provided in earbuds, headphones, a headset or hearing aids, or a computer or a mobile device, such as phone or tablet, or in the cloud, or part of an embedded system.

11. The system according to claim 9, wherein the software unit for offline and/or online processing and/or the software unit for real-time processing involve one or more dynamic filters for compensating the data, preferably multiple dynamic filters are involved for compensating the data, preferably a number of from 2-20 dynamic filters are involved for compensating the data, preferably each dynamic filter is operating with dynamically changing gain dependent on dynamically changing input signal level at the filter frequency, optionally said one or more dynamic filters are dynamically changed based on an input-signal and at least one parameter, preferably said one or more dynamic filters change the amplification depending on a change in sound pressure.

12. (canceled)

13. The system according to claim 10, wherein input sound pressure is amplified so that the experienced sound level is fully compensated.

14. The system according to claim 9, wherein the software unit for offline and/or online processing and/or the software unit for real-time processing involve a digital signal processing (DSP) unit arranged for digital signal processing of amplification data and filter center frequencies, preferably by involving one or more filter blocks.

15. The system according to claim 11, wherein each dynamic filter involved has a center frequency in a range of 100 Hz-12.8 kHz.

16. The system according to claim 11, wherein said one or more dynamic filters are wideband filters with low order, preferably second order, and wherein the method involves compensation for adjacent frequency filter boost to ensure a control of obtained aggregate gain.

17. The system according to claim 11, wherein there is provided a dynamic filter for each frequency that needs to be amplified within the hearing restoration process.

18. The system according to claim 11, wherein said one or more dynamic filters is operating with dynamically changing gain dependent on a dynamically changing input signal level at a certain filter frequency.

19. The system according to claim 11, wherein the software unit for offline and/or online processing and/or the software unit for real-time processing involve a digital signal processing (DSP) unit using one or more filter blocks comprising a band pass filter, a sound pressure detector, and a dynamic filter,
optionally the band pass filter is arranged to filter out and the detector is arranged to measure the signal level at the dynamic filter frequency and suppress sound signals present at other frequencies,
optionally the band pass filter has a low order, preferably second order, more preferably with Q below 1.

20. (canceled)

21. (canceled)

22. The system according to claim 18, wherein the band pass filter and the dynamic filter has matching bandwidths.

23. The system according to claim 11, wherein the software unit for offline and/or online processing and/or the software unit for real-time processing involve a digital signal processing (DSP) unit using one or more filter blocks comprising a gain table which uses table data, e.g. from algorithms, and translates the current input level to a gain setting in the dynamic filter,
optionally the software unit for offline and/or online processing and/or the software unit for real-time processing involves a digital signal processing (DSP) unit using multiple filter blocks.

24. (canceled)
